(19) United States
(12) Plant Patent Application Publication
(10) Pub. No.: US 2013/0074231 P1
(43) Pub. Date: Mar. 21, 2013

(54) Avocado rootstock named 'Zentmyer'

(50) ;

(71) Applicants: John A. Menge, Webster, WI (US); Gray E. Martin, Fallbrook, CA (US); Berthold O. Bergh, Riverside, CA (US); Brandon S. McKee, Riverside, CA (US); Lucille Guillemet, Chino, CA (US)

(72) Inventors: John A. Menge, Webster, WI (US); Gray E. Martin, Fallbrook, CA (US); Berthold O. Bergh, Riverside, CA (US); Fred B. Guillemet, Chino, CA (US); Brandon S. McKee, Riverside, CA (US)

(73) Assignee: The Regents of the University of California, Oakland, CA (US)

(21) Appl. No.: 13/200,179

(22) Filed: Sep. 19, 2011

(51) Int. Cl. A01H 5/00

(52) U.S. Cl. PLT/200

(57) ABSTRACT

A new and distinct variety of Persea americana tree having a high tolerance under most conditions to Phytophthora cinnamomi when used as a rootstock. However, it is severely damaged by salt and is not recommended for locations where salt is a problem. This variety does not yield well under non-root rot conditions in comparison to similar varieties, making it desirable for replant situations where root rot infested soils are a problem.

### LATIN NAME OF THE GENUS AND SPECIES

The avocado cultivar of this invention is botanically identified as Persea Americana Mill.

### VARIETY DENOMINATION

The variety denomination is ‘Zentmyer’

### BACKGROUND OF THE INVENTION

Avocado root rot is the limiting factor for the growth of avocados throughout the world. Avocado root rot is caused by the fungus Phytophthora cinnamomi, which attacks and kills the feeder roots of avocado trees. The resultant lack of roots causes the tree to eventually die from water stress. There are a number of varieties of rootstocks that have some tolerance to the disease. These varieties included ‘Duke 7’ (unpatented), the most commonly planted tolerant rootstock in the world; and ‘Thomas’ (U.S. Plant Pat. No. 6,628), another root rot tolerant rootstock. However, even with these rootstocks, growers must still use a variety of methods, including mounding, mulching and the applications of chemical fungicides, to keep the tress from dying in many soils. More resistant rootstocks are necessary to eliminate avocado root rot as a major disease threat.

Screening and greenhouse evaluation of rootstocks

‘Zentmyer’ was identified and characterized using the following screening protocol. As it is difficult to breed avocados because only one in approximately one thousand flowers actually set fruit, plant breeding blocks of avocados were isolated to prevent out crossing with susceptible rootstocks. The breeding blocks were made up of various combinations of selected rootstocks including, ‘Thomas’ (U.S. Plant Pat. No. 6,628), ‘Barr Duke’ (U.S. Plant Pat. No. 6,627), ‘G6’, ‘Duke 7’, ‘Duke 9’, ‘UC 2001’, ‘UC 2011’, ‘Toro Canyon’ (U.S. Plant Pat. No. 5,642), ‘Spencer’, ‘CR1-71’, ‘G 810’, ‘G 875’, ‘G 755A’, ‘VC 256’, and ‘Steyemarkii’. In order to synchronize blooming, attempts were made to girdle late-blooming varieties and spray early-blooming varieties with the pesticide Unicona-zole-P.

Initial screening was carried out by germinating seeds, which were harvested from the breeding blocks, in flats of vermiculite in the greenhouse. Phytophthora cinnamomi-infested millet was placed in rows along with the young roots of the test seedlings. After 8-10 weeks roots were evaluated and those with a high percentage of surviving roots were transplanted to soil mix incorporated with P. cinnamomi-infested millet. Rootstocks that survived this test were planted and grown in P. cinnamomi-infested soils. Survivors were examined more carefully for various types of resistance using clonally propagated material.
a. Root survival—Rootstocks were grown in typical California avocado soils, inoculated with P. cinnamomi and evaluated for growth, root length and percent healthy roots.
b. Root regeneration—Rootstocks were grown in soil inoculated with P. cinnamomi, treated with Aliette to halt Phytophthora root rot and evaluated for root regeneration.
c. Attraction to P. cinnamomi—Roots of the rootstocks were placed in water baths with motile zoospores of P. cinnamomi. The numbers of spores attracted to the roots were evaluated.

Rootstocks that performed well in the screening and greenhouse evaluations were further tested under field conditions.

### Selection of ‘Zentmyer’

‘Zentmyer’ was developed at Riverside, Calif. The maternal parent is ‘Thomas’ (U.S. Plant Pat. No. 6,628) avocado variety. The pollen parent is unknown. Specifically, the ‘Zentmyer’ rootstock variety was selected in 1993 from an agricultural operations land located Riverside, Calif. The fruit were collected from the avocado breeding blocks, the seed removed, and planted in vermiculite. The seeds were grown in a greenhouse. The plants were inoculated with the fungus Phytophthora cinnamomi. After showing tolerance to the disease, ‘Zentmyer’ was selected as a single plant for further testing. Budwood was collected from the plants and grafted to the stumps of adult avocado trees that had been cut down at Irvine, Calif. The new varieties grew into trees which provided budwood for further testing. At least two ‘mother’ trees of the variety are growing in Irvine Calif., along with the germplasm. During screening and evaluation, ‘Zentmyer’, which was selected and originally designated ‘PP4’, distinguished itself from other varieties by having a high tolerance against Phytophthora root rot. The properties of ‘Zentmyer’ were found to be true to type and transmissible by asexual reproduction.

### BRIEF SUMMARY OF THE INVENTION

This invention relates to a new and distinct avocado variety. ‘Zentmyer’ is an avocado tree having a rootstock that has a high tolerance against Phytophthora root rot under most conditions. However, it is severely damaged by salt and is not recommended for locations where salt is a problem. This variety also does not yield well under non-root rot conditions in comparison to similar varieties. For these reasons it may be an excellent choice for replant situations where root rot infested soils are a problem.

### BRIEF DESCRIPTION OF THE DRAWINGS

FIG. 1 illustrates a nine-year-old top-worked tree of the ‘Zentmyer’ variety while growing in Irvine, Calif.

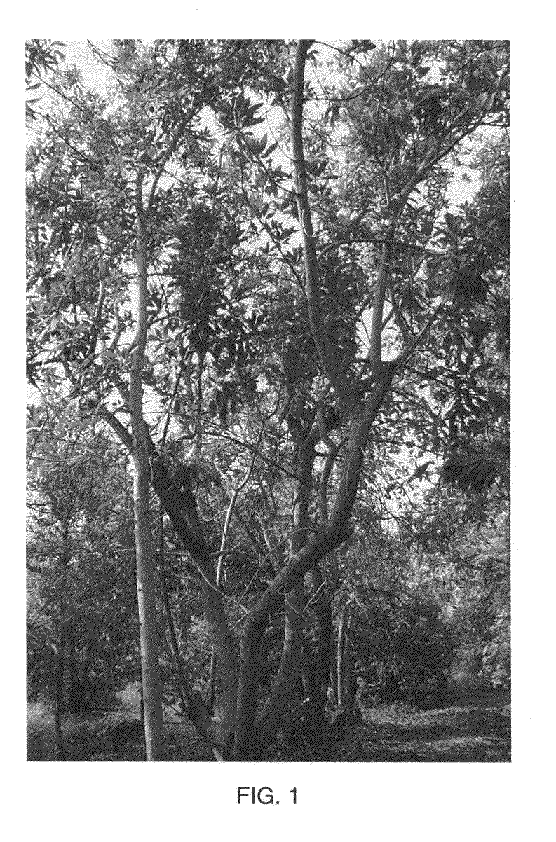

FIG. 2 illustrates typical mature foliage of the ‘Zentmyer’ variety with dimensions in centimeters shown at the right.

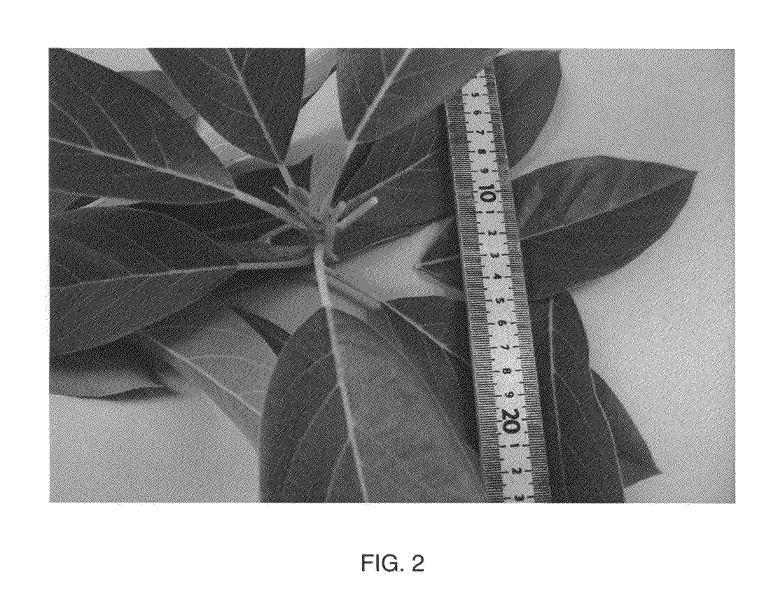

FIG. 3 illustrates typical flush foliage of the ‘Zentmyer’ variety with dimensions in centimeters shown at the bottom.

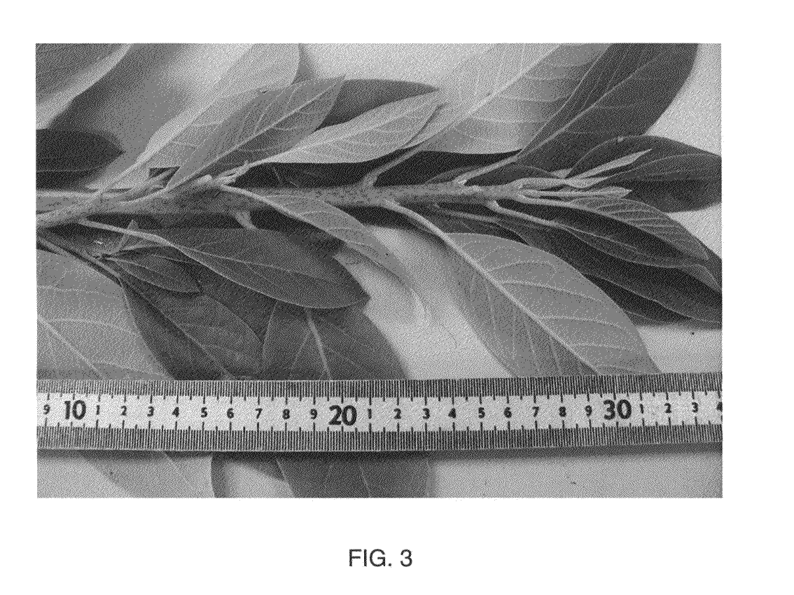

FIG. 4a illustrates typical inflorescence with dimensions in centimeters shown at the right and FIG. 4b illustrates typical inflorescence by itself.

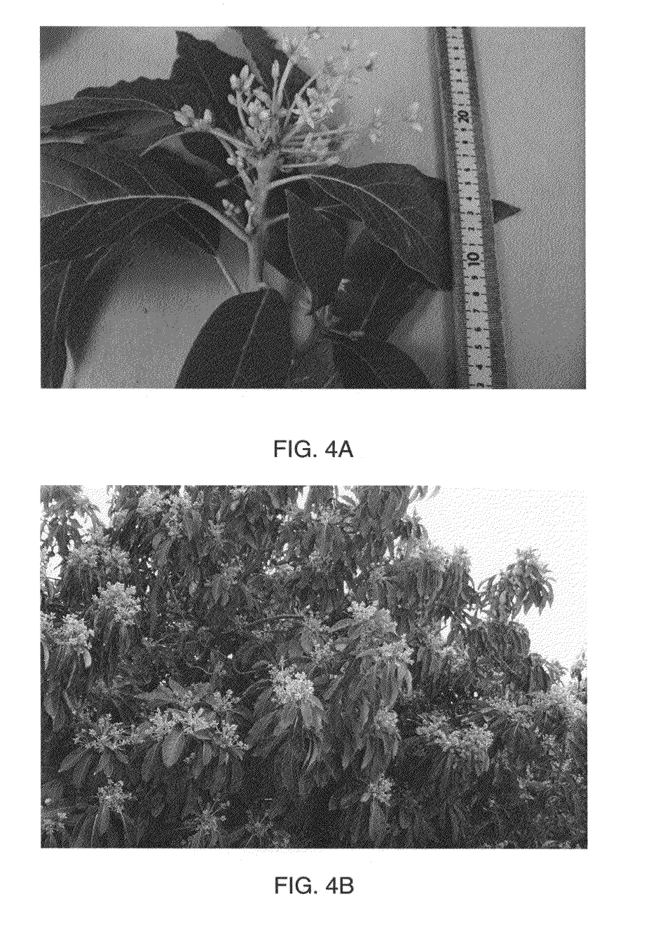

FIG. 5 illustrates a typical external view of the fruit of the ‘Zentmyer’ variety, with dimensions in centimeters shown at the bottom.

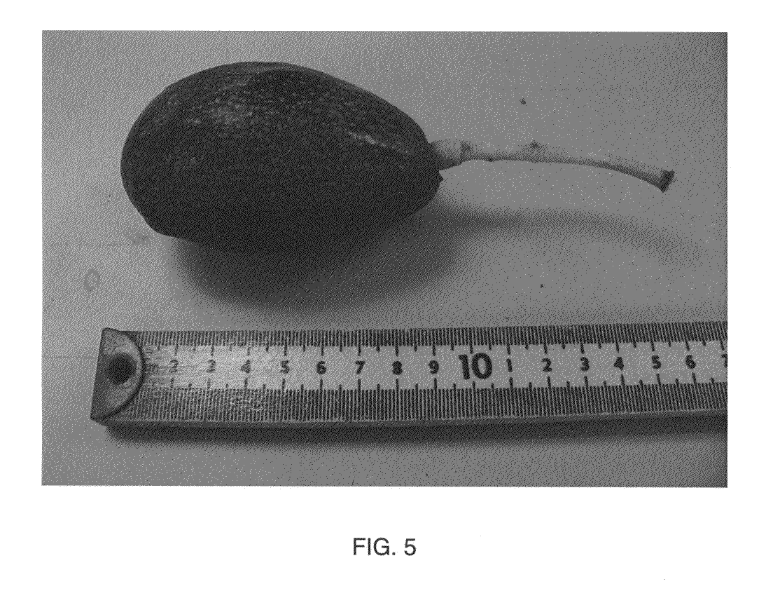

FIG. 6 illustrates typical internal views of the fruit of the ‘Zentmyer’ variety, with and without the seed. Dimensions in centimeters are shown at the bottom.

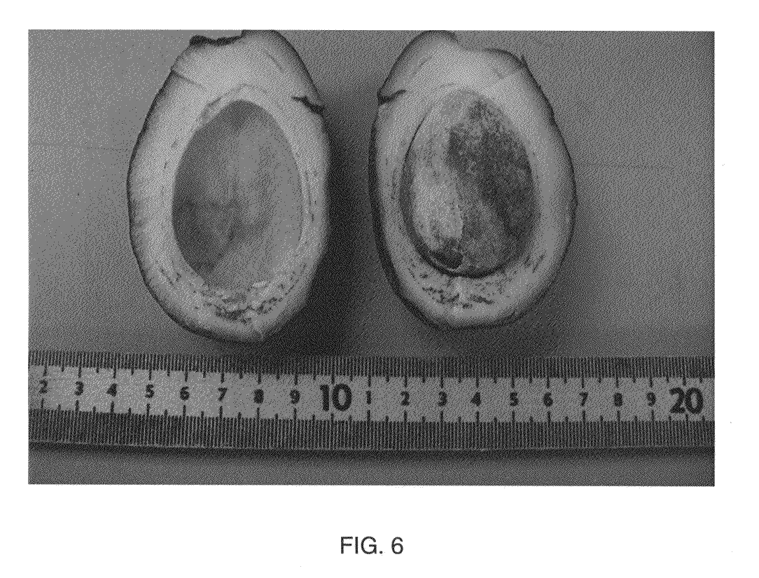

### DETAILED DESCRIPTION OF THE INVENTION

The following is a detailed description of the new ‘Zentmyer’ variety, which was taken from an approximately nine-year-old mature tree, with the exception as a rootstock for a specific scion when reference is made to root rot resistance and salinity tolerance. The tree is located in a experimental orchard in Irvine, Calif. and is grafted on a Persea americana seedling used as a rootstock.

The Royal Horticulture Society (R.H.S.) color numbering system is used herein for the color description of the rind, seed, bark, leaf, flower, flesh color and other interest of the ‘Zentmyer’ avocado tree.
Trees, foliage, and flowers:
Tree:
   Growth habit.—vigorous, upright and spreading when compared to the rootstock ‘Thomas’.
   Vigor.—below are data on the vigor of ‘Hass’ grafted onto the rootstock of ‘Zentmyer’, as determined by trunk diameter measurements from trees planted in an orchard with Phytophthora cinnamomi in Escondido Calif.

TABLE 1
Trunk diameter (cm)

| Rootstock | year 1 | year 2 | year 3 | year 4 | year 5 |
|---|---|---|---|---|---|
| PP#4 Zent. | 2.40 | 4.39 | 7.12 | 9.20 | 11.25 |
| Thomas | 2.44 | 4.29 | 6.75 | 8.40 | 10.84 |

* Malone ranch, Escondido Ca., with Hass scion

TABLE 2
Canopy volume (cubic feet)

| Rootstock | year 1 | year 2 | year 3 | year 4 | year 5 |
|---|---|---|---|---|---|
| PP#4 Zent. | 14.81 | 77.27 | 397.4 | 410. | 1573 |
| Thomas | 13.56 | 84.48 | 388.5 | 367. | 1076 |

*Malone ranch, Escondido Ca., with Hass scion

Size.—medium. The typical canopy size of a three year old top-worked ‘Thomas’ is 388 cu. ft. By comparison the canopy size of a three year old top-worked ‘Zentmyer’ is 397 cu. ft.
Branch:
   Color.—the color of the one year old branch is green (RHS 144D).
   Smoothness.—the bark of a one year old branch is smooth.
   Lenticels.—the lenticles of a one year old branch are conspicuous.
Main stem:
   Color.—grayed-green (RHS 197A and RHS 197D).
   Texture of bark.—corky.
Young shoot (flush):
   Intensity of anthocyanin coloration.—weak.
   Color.—grayed-orange (RHS 166A).
   Conspicuousness of lenticles.—medium.
   Color of lenticels.—purple (RHS 185B).
   Size of lenticels.—1.0 mm long.
   Concentration of lenticels.—+/−26 lenticels per square cm.
   Color of upper side.—grayed-orange (RHS 174A).
   Glossiness of upper side.—medium.
   Color of lower surface.—grayed-orange (RHS 177A).
Mature leaf:
   Length.—15.0 cm.
   Width.—6.0 cm.
   Ratio length/width.—2.5.
   Shape.—lanceolate.
   Color of upper side.—green (RHS 137A).
   Color of lower side.—green (RHS 138B).
   Glossiness of upper side.—medium.
   Prominence of veins on lower side.—prominent and in relief.
   Color of veins.—yellow-green (RHS 151A).
   General shape and cross-section.—concave.
   Reflexing of apex.—absent.
   Color of petiole.—yellow-green (RHS 144A).
   Anise aroma.—absent.
   Margin.—leaf margin is very weak.
   Leaf apex shape.—acuminate.
   Leaf base shape.—lanceolate.
   Length of leaf petiole.—approximately 3.0 cm.
   Leaf arrangement.—upright.
Flower:
   Bud size.—approximately 5 mm in length and approximately 4 mm in diameter.
   Bud shape.—ovoid.
   Bud color.—yellow-green (RHS 153A).
   Opening.—belongs to group “A”, male opening (i.e. with mature stamens) occurs in the afternoon, the flower closes over night, and female opening (i.e. with mature pistil) occurs the next morning; the flower's opening cycle lasts 20-24 hours.
   Petals.—borne in two whorls of three perianth lobes. The petals possess entire margins and petal coloration is near yellow-green (RHS N144B).
   Stamen.—there are commonly nine fertile stamens with each having two basal orange nectar glands and three staminodia. The anthers are tetrathecal.
   Pistil.—the single pistil with a slender style and small stigmatic surface has one carpel with one ovule. The ovary is superior.
   Pedicel.—commonly approximately 7 mm in length and approximately 1.9 mm in diameter. The coloration is near yellow-green (RHS N144A).
   Number of flowers on inflorescence.—approximately 170-200 flowers per inflorescence.
   Fragrance.—absent.
   Bloom.—bloom period at Riverside, Calif. experiment station varies with cultural conditions. On average ‘Zentmyer’ has been found to bloom from 1st of February through 20th of March.
Fruit, fruit and production characteristics:
Fruit:
   Length.—9.5 cm.
   Width.—5.5 cm.
   Ratio length/width.—1.7.
   Shape.—oblong.
   Color of skin (when ripe).—yellow-green (RHS 144A) with some patches of purple (RHS N79).
   Texture of skin.—smooth.
   Presence of longitudinal ridges.—absent.
   Thickness of skin.—thin.
   Adherence of skin to flesh.—strong.
   Main color of flesh.—yellow-green (RHS 154C).
   Color of intensely colored area of flesh next to skin.—green (RHS 140A).
   Width of intensely colored area next to skin.—3.0 mm.
   Conspicuousness of fibers in flesh.—inconspicuous.
Seed:
   Length.—5.6 cm.
   Width.—3.4 cm.
   Shape (in longitudinal section).—elliptical.
   Shape (in cross section).—circular.
   Color of seed coat (fresh).—grayed-orange (RHS 166B).
   Time of harvesting.—‘Zentmyer’ fruit ripen in September (in Riverside Calif.).
   Resistance to pests.—strong resistance to Phytophthora cinnamomi.
   Tolerance to salinity.—sensitive to salinity.
   Market use.—the fruit of ‘Zentmyer’ are not intended for market use, but rather the variety is used as a rootstock onto which commercial varieties, such as ‘Hass’ are grafted.

TABLE 3
Rootstock rating at San Ron Ranch,
Santana, Ventura County, August 2001¹

| | Tree rating | | Canopy | | Trunk | | No. |
|---|---|---|---|---|---|---|---|
| | (0-5; | | volume | | diameter | | trees |
| Rootstock | 5 = dead) | | (cu ft) | | (cm) | | dead |
| Steddom | 0.80 | a | 13.89 | a | 1.92 | a | 1 |
| Merensky II | 0.90 | a | 15.10 | a | 1.48 | a | 1 |
| Uzi | 0.90 | a | 16.92 | a | 2.02 | a | 0 |
| Zentmyer | 1.05 | a | 16.48 | a | 2.05 | a | 1 |
| G755A (Brokaw) | 1.65 | a | 5.55 | a | 1.62 | a | 1 |
| Medina | 1.90 | a | 12.66 | a | 1.70 | a | 2 |
| Berg | 2.20 | a | 13.80 | a | 1.29 | a | 4 |
| McKee | 2.35 | a | 9.05 | a | 1.52 | a | 1 |
| Duke 7 | 2.50 | a | 11.40 | a | 1.24 | a | 4 |
| Thomas | 2.65 | a | 10.22 | a | 1.15 | a | 4 |
| G755 A (C&M) | 2.75 | a | 11.66 | a | 1.49 | a | 2 |
| UC 2023 | 3.00 | a | 6.21 | a | 1.25 | a | 3 |

¹Mean values in each column followed by identical letters are not statistically different according to Waller's k-ratio t test.

TABLE 4
Rootstock rating at San Ron Ranch, Santana, Ventura County, November 2002.
Two-year trial to-date.

| | Tree rating | | Canopy | | Trunk | | Fruit rating | | Tip burn | | Canker | | No. |
|---|---|---|---|---|---|---|---|---|---|---|---|---|---|
| | (0-5; | | volume | | diameter | | (0-5; | | rating | | rating | | trees |
| Rootstock | 5 = dead) | | (cu ft) | | (cm) | | 5 = heavy) | | (0-5) | | (0-5) | | dead |
| Merensky II | 0.17 | d | 72.27 | abc | 3.49 | ab | 0.78 | bcd | 0.00 | a | 0.33 | a | 0/9 |
| Uzi | 0.50 | cd | 69.64 | abcd | 3.64 | a | 2.50 | a | 0.33 | a | 0.00 | a | 1/10 |
| Steddom | 1.00 | bcd | 67.95 | abcd | 2.94 | abc | 1.70 | abc | 0.25 | a | 0.00 | a | 2/10 |
| Medina | 1.06 | bcd | 79.89 | ab | 3.26 | ab | 0.00 | d | 0.75 | a | 0.00 | a | 1/9 |
| Zentmyer | 1.50 | bcd | 81.44 | a | 3.19 | ab | 0.60 | bcd | 0.38 | a | 0.63 | a | 1/10 |
| Duke 7 | 1.67 | bcd | 32.48 | abcde | 2.31 | abcd | 1.11 | abcd | 0.38 | a | 0.38 | a | 3/9 |
| Berg | 1.72 | bcd | 46.57 | abcde | 2.21 | abcd | 2.00 | ab | 0.17 | a | 0.83 | a | 3/9 |
| McKee | 1.78 | abcd | 30.92 | bcde | 2.24 | abcd | 0.22 | cd | 0.43 | a | 0.29 | a | 2/10 |
| G755A (Brokaw) | 2.30 | abcd | 19.98 | de | 1.90 | bcd | 0.10 | d | 0.29 | a | 0.14 | a | 3/10 |
| Thomas | 2.60 | abc | 31.50 | bcde | 2.02 | abcd | 0.30 | cd | 0.17 | a | 1.00 | a | 4/10 |
| UC 2023 | 2.95 | ab | 25.50 | cde | 1.41 | cd | 0.20 | d | 0.00 | a | 0.00 | a | 5/10 |

| | | | | | | | | | | | |
|---|---|---|---|---|---|---|---|---|---|---|---|
| G755A (C&M) | 4.00 | a | 15.71 | e | 0.82 | d | 0.00 | d. | — | — | 8/10 |

TABLE 5
Tajiquas Ranch {Leo Curillo) rootstock rating, December 2003.
Three-year trial to-date.

| | Tree rating | | Canopy | | Trunk | | Fruit rating | | Salt rating | | Canker | | No. |
|---|---|---|---|---|---|---|---|---|---|---|---|---|---|
| | (0-5; | | vol | | diam | | (0-5; | | (0-5; | | rating (0-5; | | trees |
| Rootstocks | 5 = dead) | | (cu ft) | | (cm) | | 5-heavy) | | 5 = severe) | | 5-severe) | | dead (%) |
| Zentmyer | 0.313 | d | 48.0 | ab | 6.45 | a | 1.75 | abc | 0.00 | a | 0.00 | a | 0 |
| Merensky II | 0.556 | cd | 71.6 | a | 6.49 | a | 2.67 | a | 0.00 | a | 0.00 | a | 0 |
| Steddom | 0.677 | bcd | 47.2 | ab | 5.18 | ab | 2.00 | ab | 0.00 | a | 0.06 | a | 6 |
| Parida | 1.147 | abcd | 50.6 | ab | 4.91 | ab | 1.53 | abcd | 0.00 | a | 0.07 | a | 18 |
| Evstro | 1.353 | abcd | 49.6 | ab | 5.55 | ab | 2.29 | ab | 0.00 | a | 0.06 | a | 0 |
| Merensky I | 1.441 | abcd | 48.6 | ab | 5.01 | ab | 1.41 | bcd | 0.00 | a | 0.06 | a | 18 |
| Guillemet | 1.588 | abc | 39.6 | b | 4.58 | b | 0.41 | d | 0.00 | a | 0.08 | a | 22 |
| Thomas | 1.875 | ab | 43.4 | ab | 4.45 | b | 0.72 | cd | 0.00 | a | 0.08 | a | 29 |
| UC 2023 | 2.188 | a | 27.2 | b | 4.07 | b | 0.31 | d | 0.08 | a | 0.00 | a | 19 |
| VC 207 | 2.382 | a | 32.4 | b | 3.79 | b | 1.12 | bcd | 0.00 | a | 0.00 | a | 35 |

Mean values in each column followed by identical letters are not statistically different according to Waller's k-ratio t test.

TABLE 6
Rootstock ratings of avocado trees planted in root
rot soil at Malone Ranch Plot 1, Escondido, July 2002

| | Tree | | | | | | Fruit set | | | | |
|---|---|---|---|---|---|---|---|---|---|---|---|
| | rating | | Canopy | | Trunk | | rating | | Tip | | |
| | 0-5; | | volume | | diameter | | 0-5; | | Burn | Cankers | Dead |
| Rootstocks | 5 = dead | | Cu ft | | Cm | | 5 = heavy | | Number trees affected | | |
| Zentmyer | 0.00 | c | 397.4 | abc | 7.12 | bcd | 1.53 | cd | 0 | 0 | 0/15 |
| Rio Frio | 0.00 | c | 313.5 | cdef | 6.33 | cdef | 2.13 | bcd | 0 | 0 | 0/16 |
| Merens I | 0.00 | c | 543.6 | a | 8.74 | a | 3.50 | a | 0 | 0 | 0/14 |
| Merensk II | 0.02 | c | 409.0 | abc | 7.81 | abc | 2.84 | ab | 0 | 1 | 0/17 |
| VC 241 | 0.06 | c | 238.4 | defg | 6.19 | defg | 1.41 | cd | 0 | 0 | 0/16 |
| Uzi | 0.29 | bc | 504.3 | ab | 8.57 | ab | 2.76 | ab | 2 | 0 | 1/17 |
| Steddom | 0.36 | bc | 376.1 | bcde | 7.07 | bcd | 2.43 | bc | 0 | 0 | 1/14 |
| Thomas | 0.44 | bc | 388.5 | bcd | 6.75 | cde | 1.12 | de | 0 | 0 | 1/17 |
| Guillemet | 0.59 | bc | 192.0 | fgh | 4.90 | fgh | 1.12 | de | 3 | 1. | 2/17 |
| Spencer sdlg | 0.63 | bc | 225.8 | efg | 5.24 | efgh | 1.56 | cd | 0 | 0 | 2/16 |
| Leo | 0.67 | bc | 288.2 | cdef | 5.89 | defgh | 1.60 | cd | 0 | 0 | 2/15 |
| Spencer clonal | 0.69 | bc | 163.8 | fgh | 4.65 | gh | 1.54 | cd | 0 | 0 | 5/16 |
| Duke 7 | 1.00 | b | 129.3 | gh | 4.38 | h | 1.47 | cd | 0 | 0 | 3/15 |
| G755A | 0.16 | b | 294.1 | cdef | 5.86 | defgh | 1.56 | cd | 2 | 1 | 3/16 |
| PolyN | 4.12 | a | 65.6 | h | 1.26 | i | 0.24 | e | 0 | 0 | 14/17 |

TABLE 7
Malone Field 1 rootstock trial tree ratio
April 2003¹. Four-year trial to-date

| | Tree rating | | Canopy | | Trunk | | | | Canker | | | | Dead |
|---|---|---|---|---|---|---|---|---|---|---|---|---|---|
| | (0-5; | | volume | | diam. | | | | (0-5; | | Fruit | | trees |
| Rootstock | 5 = dead) | | (cu ft) | | (cm) | | Salt | | 5 = heavy) | | rating² | | (%) |
| MerenI | 0.00 | d | 551 | ab | 10.7 | a | 0.08 | cd | 0 | a | 2.97 | abc | 0 |
| VC241 | 0.06 | d | 281 | efgh | 8.0 | abc | 0.03 | cd | 0 | a | 3.41 | ab | 0 |
| Rio Frio | 0.07 | d | 362 | efcd | 8.7 | abc | 0.00 | d | 0 | a | 3.73 | a | 0 |
| Zentmyer | 0.07 | d | 410 | bcde | 9.2 | ab | 0.32 | bc | 0 | a | 3.71 | a | 0 |
| MerenII | 0.18 | d | 532 | abc | 9.4 | ab | 0.21 | dc | 0.1 | a | 2.97 | abc | 0 |
| Spen sdlg | 0.36 | d | 263 | efgh | 6.9 | bc | 0.00 | d | 0 | a | 3.57 | ab | 7 |
| Uzi | 0.38 | d | 669 | a | 10.6 | a | 0.68 | a | 0 | a | 3.47 | ab | 6 |
| Steddom | 0.39 | d | 478 | bcd | 8.6 | abc | 0.32 | bc | 0 | a | 3.75 | a | 7 |
| Thomas | 0.47 | cd | 367 | cdef | 8.4 | abc | 0.62 | ab | 0 | a | 3.53 | ab | 6 |
| Leo | 0.77 | cbd | 274 | efgh | 7.3 | abc | 0.13 | cd | 0 | a | 3.29 | ab | 13 |
| Guillemet | 0.83 | cbd | 190 | ghi | 6.2 | bc | 0.13 | cd | 0 | a | 2.90 | abc | 13 |
| Duke7 | 1.34 | cb | 127 | hi | 8.8 | abc | 0.16 | cd | 0 | a | 1.53 | de | 19 |
| Spen cl | 1.44 | b | 211 | fghi | 5.3 | c | 0.12 | cd | 0 | a | 2.35 | bcd | 23 |
| G755A | 1.69 | b | 322 | defg | 7.0 | bc | 0.25 | cd | 0 | a | 1.78 | cd | 25 |
| PolyN | 4.15 | a | 77 | i | 1.5 | d | 0.06 | cd | 0 | a | 0.29 | e | 82 |

¹Mean values in each column followed by identical letters are not statistically different according to Waller's k-ratio t test.
²Fruit was rated in November 2003.

TABLE 8
Malone Ranch Plot 1, Temecula,
yield 2003 ^{1;2}. Four year trial to-date.

| | Fruit weight/ | | Number | | Fruit | |
|---|---|---|---|---|---|---|
| Rootstock | tree (kg) | | fruit/tree | | weight (kg) | |
| Zentmyer | 15.89 | a | 68.64 | a | 0.219 | a |
| Uzi | 13.99 | ab | 59.24 | ab | 0.19 5 | ab |
| Spencer seedling | 12.52 | ab | 56.27 | ab | 0.181 | ab |
| Merensky II | 11.83 | ab | 51.12 | ab | 0.185 | ab |
| Rio Frio | 10.87 | abc | 51.33 | ab | 0.187 | ab |
| Steddom | 10.01 | abc | 46.20 | abc | 0.175 | abc |
| Thomas | 8.50 | abcd | 40.12 | abcd | 0.154 | abc |
| G755A | 8.08 | abcd | 34.56 | abcd | 0.116 | bc |
| VC241 | 7.44 | bcd | 31.75 | bcd | 0.202 | ab |
| Guillemet | 7.42 | bcd | 30.00 | bcd | 0.196 | ab |
| Spencer clonal | 6.99 | bcd | 32.00 | bcd | 0.136 | abc |
| Merensky I | 6.95 | bcd | 32.08 | bcd | 0.148 | abc |
| Leo | 6.53 | bcd | 28.14 | bcd | 0.140 | abc |
| Duke 7 | 3.33 | cd | 14.81 | cd | 0.138 | abc |
| PolyN | 1.72 | d | 5.71 | d | 0.076 | c |

¹ Mean values in each column followed by identical letters are not statistically different according to Waller's k-ratio t test.
² Only fruit which were grade size were picked; remaining fruit on trees to be picked later.

TABLE 9
Malone II, Escondido, Tree ratings, July 2002

| | Tree rating | Canopy vol. | Trunk | No. trees | No. trees | No. trees |
|---|---|---|---|---|---|---|
| Rootstock | (0-5; 5 = dead) | (cu ft) | diam (cm) | Dead | w/tip burn | w/canker |
| Uzi | 0.039 b | 34.69 a | 2.43 a | 0 | 6 | 0 |
| Guillemet | 0.042 b | 22.86 a | 2.06 a | 0 | 4 | 0 |
| Zentmyer | 0.077 b | 22.40 a | 2.25 a | 0 | 2 | 0 |
| Spencer sdlg | 0.536 b | 27.81 a | 2.01 a | 0 | 2 | 1 |
| Steddom | 0.615 b | 18.93 a | 1.99 a | 1 | 0 | 0 |
| Berg | 0.714 b | 21.42 a | 1.98 a | 0 | 1 | 2 |
| Merensky II | 0.750 b | 32.07 a | 2.10 a | 2 | 0 | 1 |
| Elinor | 0.786 b | 29.44 a | 2.03 a | 1 | 0 | 2 |
| Thomas | 0.846 b | 23.07 a | 1.85 a | 1 | 2 | 0 |
| Pond | 1.00 ab | 30.55 a | 2.15 a | 1 | 0 | 2 |
| Crowley | 1.083 ab | 23.78 a | 1.86 a | 2 | 1 | 0 |
| G755A | 1.231 ab | 22.64 a | 1.85 a | 2 | 0 | 0 |
| Duke 9 | 2.270 a | 9.40 a | 1.07 b | 5 | 0 | 0 |

There were significant differences at P = 0.01 between blocks for all tree parameters analyzed.

TABLE 10
Malone H, tree ratings, April 2003. Two-year trial to-date.

| | Tree rating | Canopy | Trunk | Fruit rating | Salt rating | Canker rating | |
|---|---|---|---|---|---|---|---|
| | (0- | vol | diam | (0-5; | (0-5; | (0-5;5 = | No. trees |
| Rootstock | 5;5 = dead) | (cu ft) | (cm) | 5 = heavy) | 5 = severe) | severe) | Dead (%) |
| Uzi | 0.267 c | 88.76 a | 4.193 a | 0.0 a | 0.933 ab | 0.000 a | 0 |
| Berg | 0.531 c | 44.16 a | 2.956 bc | 0.0 a | 0.633 abcd | 0.000 a | 6 |
| Zentmyer | 0.600 c | 54.37 a | 3.393 ab | 0.0 a | 1.000 a | 0.000 a | 7 |
| Merensky II | 0.833 bc | 68.49 a | 3.333 ab | 0.0 a | 0.154 cd | 0.308 a | 13 |
| Steddom | 0.867 bc | 56.42 a | 3.127 ab | 0.0 a | 0.321 bcd | 0.286 a | 7 |
| Pond | 0.906 bc | 55.05 a | 3.188 ab | 0.0 a | 0.767 abc | 0.200 a | 6 |
| Spenser sdlg | 0.906 bc | 51.45 a | 2.988 bc | 0.0 a | 0.300 bcd | 0.200 a | 6 |
| Crowley | 0.964 bc | 42.05 a | 3.021 bc | 0.0 a | 0.083 d | 0.000 a | 14 |
| Thomas | 1.071 bc | 49.99 a | 2.900 bc | 0.0 a | 0.731 abc | 0.000 a | 0 |
| Guillemet | 0.167 abc | 43.64 a | 2.960 bc | 0.1 a | 0.615 abcd | 0.133 a | 13 |
| Elinor | 1.393 abc | 58.40 a | 2.864 bc | 0.0 a | 0.333 bcd | 0.167 a | 14 |
| G755A | 2.156 ab | 44.21 a | 2.819 bc | 0.0 a | 0.846 ab | 0.077 a | 13 |
| Duke 9 | 2.577 a | 32.16 a | 1.885 c | 0.0 a | 0.313 bcd | 0.500 a | 38 |

TABLE 11
Smith Ranch, Santa Paula, rootstock rating, December 2002

| | Tree rating | | | | Salt burn | | |
|---|---|---|---|---|---|---|---|
| | (0-5;5 = | Canopy | Trunk | | (0-5; | | Trees |
| Rootstock | dead) | vol (cu ft) | diam (cm) | Fruit set | 5-heavy) | Cankers | dead (%) |
| McKee | 0.00 b | 51.41 a | 3.45 bc | 0.00 a | 0 | 0 | 0 |
| Merensky II | 0.00 b | 53.45 a | 3.66 ab | 0.00 a | 0 | 0 | 0 |
| Pond | 0.00 b | 55.08 a | 3.69 a | 0.00 a | 0 | 0 | 0 |
| Guillemet | 0.00 b | 37.98 b | 2.71 f | 0.00 a | 0 | 0 | 0 |
| Zentmyer | 0.00 b | 51.92 a | 3.38 cd | 0.00 a | 0 | 0 | 0 |
| Thomas | 0.00 b | 36.66 b | 3.15 de | 0.00 a | 0 | 0 | 0 |
| Crowley | 0.03 b | 34.91 b | 3.17 d | 0.05 a | 0 | 0 | 0 |
| Duke 9 | 0.05 b | 31.93 b | 2.93 ef | 0.00 a | 0 | 0 | 0 |
| Steddom | 0.27 a | 37.14 b | 2.75 f | 0.00 a | 0 | 0 | 0 |

Mean values in each column followed by identical letters are not statistically different according to Waller's k-ratio.

TABLE 12
Smith Ranch, Santa Paula, rootstock rating, December 2003. Two-year trial
to-date.

| | | | Trunk | | Salt burn | | |
|---|---|---|---|---|---|---|---|
| | Tree rating | Canopy vol | diam | Fruit | (0-5; | | Trees dead |
| Rootstock | (0-5;5 = dead) | (cu ft) | (cm) | set | 5-heavy) | Cankers | (%) |
| McKee | 0.025b | 184.1b | 5.88bc | 1.90ab | 0 | 0 | 0 |
| Merensky II | 0.000b | 246.8a | 6.18abc | 2.60a | 0 | 0 | 0 |
| Pond | 0.000b | 192.0b | 6.24ab | 0.00d | 0 | 0 | 0 |
| Guillemet | 0.000b | 118.8cd | 5.38de | 0.00d | 0 | 0 | 0 |
| Zentmyer | 0.026b | 182.8b | 6.41a | 1.32bc | 0 | 0 | 0 |
| Thomas | 0.237a | 174.9b | 5.72cd | 0.47cd | 0 | 0 | 0 |
| Crowley | 0.150ab | 124.7c | 5.42de | 2.15ab | 0 | 0 | 0 |
| Duke 9 | 0.053ab | 132.6c | 5.19e | 1.89ab | 0 | 0 | 0 |
| Steddom | 0.083ab | 86.3d | 5.00e | 2.00ab | 0 | 0 | 0 |

Mean values in each column followed by identical letters are not statistically different according to Waller's k-ratio t test . . .

TABLE 13
Weaver Ranch, Temecula rootstock ratings, Sept 2002

| | Tree rating | Canopy | Trunk | Fruit rating | Salt | Cankers | |
|---|---|---|---|---|---|---|---|
| | (0-5; | vol. | diam | (0-5; | damage (0- | (0-5; | No. trees |
| Rootstock | 5 = dead) | (cu ft) | (cm) | 5 = heavy) | 5; 5 = heavy) | 5 = heavy) | dead |
| Zentmyer | 0.400 c | 40.70 ab | 2.79 a | 0.00 b | 1.50 ab | 0.00 a | 0/15 |
| Crowley | 0.618 c | 40.38 ab | 2.86 a | 0.00 b | 1.34 b | 0.00 a | 1/17 |
| Elinor | 0.824 c | 40.52 ab | 2.54 a | 0.00 b | 1.59 ab | 0.00 a | 1/17 |
| Guillemet | 0.882 bc | 39.13 ab | 2.42 a | 0.00 b | 1.41 b | 0.00 a | 2/17 |
| Steddom | 0.969 bc | 29.20 bc | 2.13 ab | 1.16 a | 1.54 ab | 0.50 a | 2/16 |
| Thomas | 0.969 bc | 31.46 bc | 2.13 ab | 0.00 b | 1.50 ab | 0.00 a | 3/16 |
| Pond | 1.088 bc | 54.08 a | 2.78 a | 0.00 b | 1.40 b | 0.00 a | 2/17 |
| Uzi | 1.188 bc | 35.08 ab | 2.56 a | 0.00 b | 1.64 ab | 0.00 a | 2/16 |
| G755A | 2.088 ab | 37.85 ab | 2.41 a | 0.00 b | 2.50 ab | 0.36 a | 4/17 |
| Spencer sdlg | 2.906 a | 11.96 c | 1.39 b | 0.00 b | 2.63 a | 0.00 a | 4/16 |

TABLE 14
Weaver Ranch, Temecula, rootstock ratings, December 2003. Two-year trial
to-date.

| | Tree rating | Canopy | Trunk | Fruit rating | Salt damage | Cankers | Trees |
|---|---|---|---|---|---|---|---|
| | (0-5; | vol | diam | (0-5; | (0-5; | (0-5; | dead |
| Rootstock | 5 = dead) | (cu ft) | (cm) | 5 = heavy) | 5 = heavy) | 5 = heavy) | (%) |
| Zentmyer | 0.313c | 207.27a | 6.23a | 2.063a | 1.188ab | 0.000a | 0 |
| Pond | 0.906c | 307.04a | 5.75a | 1.813a | 0.321cd | 0.000a | 13 |
| Elinor | 0.912c | 170.37a | 4.80a. | 1.059a | 0.469cd | 0.000a | 6 |
| Guillemet | 1.059c | 199.37a | 5.73a | 0.882a | 0.893abc | 0.000a | 18 |
| Uzi | 1.094bc | 206.04a | 4.35a | 0.813a | 0.769abcd | 0.000a | 19 |
| Crowley | 1.250bc | 144.14a | 5.04a | 1.438a | 0.731abcd | 0.000a | 19 |
| Steddom | 1.281bc | 254.94a | 4.89a | 1.188a | 0.167d | 0.000a | 25 |
| Thomas | 1.313bc | 226.39a | 5.16a | 1.375a | 1.308a | 0.000a | 19 |
| G755A | 2.438ab | 175.55a | 5.23a | 0.625a | 1.167ab | 0.000a | 25 |
| Spencer sdlg | 2.813a | 42.12a | 2.26a | 0.519a | 0.500bcd | 0.000a | 44 |

What is claimed is:

1. A new and distinct rootstock variety of avocado tree having the characteristics substantially as described and illustrated herein.
